(19) United States
(12) Patent Application Publication
(10) Pub. No.: US 2026/0183460 A1
(43) Pub. Date: Jul. 2, 2026

(54) System and Method for Controlling Blood Oxygenation

(71) Applicant: Spectrum Medical Ltd., Gloucester, Gloucestershire (GB)

(72) Inventor: Stephen Turner, Gloucester, Gloucestershire (GB)

(21) Appl. No.: 19/130,784

(22) PCT Filed: Nov. 9, 2023
(86) PCT No.: PCT/GB2023/052932

§ 371 Date: May 16, 2025

(30) Foreign Application Priority Data
Nov. 18, 2022 (GB) 2217323.1

(51) Int. Cl. A61M 1/16

(52) U.S. Cl. CPC A61M 1/1698; A61M 1/1603; A61M 2202/0208; A61M 2202/0225

(57) ABSTRACT

A system (100, FIG. 1) for controlling blood oxygenation in a patient using an oxygenator 200 having a gas-blood interface 240. The system (100, FIG. 1) comprises a controller (150, FIG. 1) configured to receive a measured value of a physiological parameter indicative of the oxygenation of the patient's blood and calculate a difference between the measured value and a target value of the physiological parameter. The controller (150, FIG. 1) is further configured to control a gas supply apparatus (300, FIG. 1) to: supply a first oxygenation gas (242a) to a first interface region (240a) of the gas-blood interface (240) and supply a second oxygenation gas (242b) to a second interface region (240b) of the gas-blood interface (240); responsive to the measured value being above the target value, decrease the amount of oxygen to which the blood is exposed by reducing the flow rate of the second oxygenation gas (242b) to reduce the difference; and responsive to the flow rate of the second oxygenation gas (242b) being reduced to a threshold value, reduce the concentration of oxygen in and/or the flow rate of the first oxygenation gas (242a).

The present disclosure relates to systems and methods for controlling blood oxygenation in a patient. In particular, the disclosure relates to systems and methods for improved control of blood oxygen in patients experiencing low metabolic rate, low blood oxygen saturation, and/or elevated blood carbon dioxide levels.

Cardiac perfusion involves extracorporeal oxygenation of a patient's blood, for example, when a patient is unable to oxygenate their own blood by breathing. Extracorporeal oxygenation involves an oxygenator that acts in place of a patient's own lungs, such as during heart and/or lung surgery. During extracorporeal oxygenation, the oxygenator is used to control the levels of gases in the patient's blood (e.g. oxygen and carbon dioxide), which is a function that would usually be performed by a patient's own lungs. During a surgical procedure involving extracorporeal perfusion, a patient may experience variations in their requirements for delivery and/or removal of certain gases in the blood.

For example, the patient may experience variations in their metabolic rate. Variations in a patient's metabolic rate leads to variations in the consumption of oxygen from the patient's blood. For example, a patient with a healthy or normal metabolic rate will consume more oxygen from their blood than a patient who is experiencing a low metabolic rate. For example, a patient's metabolic rate may be deliberately reduced (e.g. by reducing the patient's temperature), before placing the patient into a state of circulatory arrest, for example, in order to perform surgery on the patient's heart.

In another example, a patient may experience variations in the saturation of oxygen in their blood. For example, a patient may suffer from low saturation of oxygen in the blood. Cyanotic patients are those suffering low saturation of oxygen in the blood, which typically leads to a blue discolouration of the skin. The condition more commonly occurs among babies, particularly those with congenital heart abnormalities. Such patients are at risk of detrimental effects caused by hyperoxia (i.e. over-oxygenation of the blood).

In another example, a patient may experience variations in the level of carbon dioxide in their blood. For example, during hypothermic circulatory arrest a patient is cooled in order to lower their metabolic rate. At lower temperatures, carbon dioxide dissolves more readily in the patient's blood and thus the level of carbon dioxide in the patient's blood can rise. Following this procedure, the carbon dioxide level must be decreased to avoid the potential detrimental effects of having too much carbon dioxide in the patient's blood.

In conventional approaches, such as those involving single chamber oxygenators, a clinician may modulate the flow rate of oxygenation gas and/or the concentration of oxygen across the entire oxygenator. This may have unintended and undesirable consequences for the overall performance of the oxygenator. For example, if the clinician reduces the flow rate in the entire oxygenator in order to reduce oxygen delivery, the removal of carbon dioxide and nitrogen would also be reduced. Therefore, a clinician may expose the patient to risks associated with poor control of blood gases (e.g. too high or too low levels of oxygen, carbon dioxide, and/or nitrogen). Such poor control can have detrimental consequences, such as: oxidative stress (in the case of over-oxygenation); increased risk of gaseous microemboli (in the case of increased nitrogen levels); tissue damage (in the case of under-oxygenation); or increased risk of cerebral swelling (in the case of increased carbon dioxide levels).

Even in cases where a clinician is able to modulate gas flow conditions differently for different portions of an oxygenator, incorrect operation of such an oxygenator can lead to the same detrimental consequences. The breadth of permutations available for controlling oxygenation gases in an oxygenator render it challenging for a clinician to control each and every gas level in the patient's blood. In particular, certain clinical scenarios require specific modulation of each blood gas component in order to achieve the precise desired physiological effect.

Therefore, there is a need to provide systems and methods for controlling blood oxygenation with improved versatility and that are capable of providing precise control of gases in a patient's blood in particular clinical scenarios.

It will be noted that, for accuracy and in line with convention in the medical field, values of pressure given herein are provided in units of mmHg (‘millimetre of mercury’). However, these values may be simply converted to atm (‘atmospheres’) in noting that 1 atm is equal to 760 mmHg, or to Pa (‘Pascal’) in noting that 1 mmHg is approximately equal to 133.3 Pa.

In accordance with a first aspect, there is provided a system for controlling blood oxygenation in a patient, as defined in claim 1.

The system is for controlling blood oxygenation in a patient using an oxygenator having a gas-blood interface. The gas-blood interface is configured to receive blood from the patient and to expose the blood to an amount of oxygen as the blood passes through the oxygenator to produce oxygenated blood. The gas-blood interface of the oxygenator comprises a first interface region and a second interface region that are each configured to be independently supplied with a respective oxygenation gas. The system comprises a controller. The controller is configured to receive a measured value of a physiological parameter indicative of the oxygenation of the patient's blood and calculate a difference between the measured value and a target value of the physiological parameter. The controller is further configured to control a gas supply apparatus to supply the first oxygenation gas to the first interface region and supply the second oxygenation gas to the second interface region. The controller is further configured to control a gas supply apparatus to, responsive to the measured value being above the target value, decrease the amount of oxygen to which the blood is exposed by reducing the flow rate of the second oxygenation gas to reduce the difference. The controller is further configured to control a gas supply apparatus to, responsive to the flow rate of the second oxygenation gas being reduced to a threshold value, reduce the concentration of oxygen in and/or the flow rate of the first oxygenation gas.

Advantageously, supplying oxygenation gas to both the first interface region and the second interface region allows the oxygenation in the patient's blood to be adjusted while exposing the blood to a ‘high’ amount of oxygen in the oxygenator. That is, the entirety of the gas-blood interface is supplied with oxygenation gas. The blood is thus exposed to oxygenation gas in both the first interface region and the second interface region, and thus is able to exchange gases through the entirety of the gas-blood interface. This is particularly advantageous when a patient requites a ‘normal’ or ‘healthy’ saturation of oxygen (e.g. 98.5%).

The oxygenation gas (e.g. the first oxygenation gas and/or the second oxygenation gas) may comprise a mixture of oxygen with nitrogen and/or carbon dioxide. For example, the oxygenation gas may comprise a mixture of nitrogen and oxygen (but no carbon dioxide). The concentration of oxygen in the oxygenation gas may be adjusted by adjusting the relative proportions of nitrogen, carbon dioxide, and/or oxygen in the oxygenation gas. The ‘concentration of oxygen in the oxygenation gas’ may equivalently be referred to as the ‘fraction of inspired oxygen’ (abbreviated as ‘FiO2’). The controller may be configured to control one or more valves or actuators that control the gas supply to the oxygenator in order to adjust (i.e. increase or reduce) the concentration of any or all components of the oxygenation gas supplied to each gas-blood interface of the oxygenator. Alternatively or additionally, controller may be configured to control one or more valves or actuators that control the gas supply to the oxygenator in order to adjust (i.e. increase or reduce) the flow rate of the oxygenation gas supplied to each gas-blood interface of the oxygenator. In this manner, the controller can control the composition and/or flow rate of the oxygenation gas supplied to the first gas-blood interface independently of the composition and/or flow rate of the oxygenation gas supplied to the second gas-blood interface.

Advantageously, the controller is configured to control the gas supply apparatus in a manner that has physiological benefits during surgery for treating a patient who is experiencing a low metabolic rate (for example, a patient undergoing circulatory arrest). A patient experiencing low metabolic rate has a reduced requirement for oxygenation of the blood because the patient's body will not consume as much oxygen from the blood as part of basic metabolic functions. Therefore, during extracorporeal oxygenation, the level of oxygenation in the patient's blood will increase because the patient is not consuming enough of the oxygen that is being supplied to the blood by the oxygenator.

A conventional approach in which over-oxygenation is not prevented would, in this circumstance, lead to the patient receiving too much oxygen from the oxygenator, thus leading to hyperoxia. Alternatively, an approach in which flow rate is reduced indiscriminately throughout an entire single or multi-chamber oxygenator would lead to a complete reduction in oxygenator functionality, rendering the extracorporeal oxygenation less effective. That is, reducing flow rate and/or concentration in the entire oxygenator would reduce oxygen delivery to the blood, but would also reduce the removal of carbon dioxide from the patient's blood. This, in turn, leads to a build-up of these undesirable gases in the patient's blood.

In contrast, the controller disclosed herein is able to avoid hyperoxia, whilst maintaining other oxygenator functionality (e.g. carbon dioxide removal, nitrogen removal) in a separate chamber. Initially, the controller controls the gas supply apparatus to reduce the flow rate of the second oxygenation gas, thereby reducing the amount of oxygen to which the blood is exposed. During this reduction, independent supplies of oxygenation gas are provided to the first interface region and the second interface region. Therefore, while the flow rate of the second oxygenation gas is reduced, the oxygenator is still capable of providing gas delivery and/or removal via the first oxygenation gas. In other words, oxygen delivery to the blood can be reduced by reducing the flow rate of the second oxygenation gas, but adequate removal of carbon dioxide and nitrogen can be concurrently achieved with the first oxygenation gas. In this regard, the first oxygenation gas may be regarded as a ‘sweep gas’. Similarly, the first interface region may be regarded as a ‘sweep chamber’or ‘sweep region’.

By ‘independently supplied’ it is meant that the composition (e.g. gas concentrations) and/or flow conditions (e.g. flow rate) of each respective oxygenation gas can be controlled independently for each interface region. The controller may be configured to alter the number of interface regions that are supplied with the oxygenation gas by switching on or off one or more of the independent gas supplies. For example, the controller may be configured to open or close one or more valves that allow or prevent the flow of gas from an independent gas supply into a respective interface region.

Furthermore, following the reduction of the flow rate of the second oxygenation gas to a threshold value, the concentration of oxygen in and/or the flow rate of the first oxygenation gas can then be reduced to further reduce the oxygen delivery as required, thereby allowing lower oxygen delivery so as to meet the target value of oxygenation for a patient with a lower metabolic rate.

For example, the controller may be configured to control the gas supply apparatus to reduce the concentration of oxygen in and/or the flow rate of the first oxygenation gas responsive to the flow rate of the second oxygenation gas being reduced to a threshold value and responsive to the measured value continuing to be above the target value.

The first and second interface regions may be arranged successively with respect to the direction of blood flow through the oxygenator. That is, as the blood passes through the oxygenator, the blood passes through each interface region, one after the other. For example, the interface regions may be arranged such that the blood passes through the second interface region before passing through the first interface region.

The first interface region and the second interface region may collectively constitute the entirety of the gas-blood interface. In other words, the gas-blood interface may be divided into only two interface regions. It will therefore be appreciated that for whatever proportion of the gas-blood interface forms the first interface region, the remaining proportion of the gas-blood interface will form the second interface region. The first interface region may be smaller than, larger than, or equal in size to the second interface region.

For example, the gas-blood interface may be divided in half, such that each of the first interface region and the second interface region comprises 50% of the gas-blood interface (i.e. a ‘50/50 split’). In another example, the first interface region may comprise 40% of the gas-blood interface and the second interface region may comprise 60% of the gas-blood interface (i.e. a ‘40/60 split’), or vice versa (i.e. a ‘60/40 split’). Advantageously, the first interface region comprising 40% of the gas-blood interface provides appropriate regulation (e.g. addition, removal, and/or maintenance) of carbon dioxide when the first interface region is provided with an oxygenation gas at a standard flow rate (e.g. the oxygenation gas flow rate being approximately the same as the blood flow rate through the oxygenator, which may be around, for example, 4-5 litres per minute) that is commonly used during perfusion procedures. Other possible divides are also contemplated herein, such as: a 30/70 split, a 70/30 split, a 25/75 split, a 75/25 split, a 20/80 split, an 80/20 split, a 10/90 split, or a 90/10 split.

The term ‘gas-blood interface’ refers to a component of the oxygenator that permits gas exchange between a gas supplied to the gas-blood interface and blood supplied to the gas-blood interface. The gas-blood interface may comprise one or more hollow fibre groups. Each hollow fibre group may comprise a plurality of hollow fibres. Each hollow fibre group may take the form of a hollow fibre bundle, a hollow fibre mat, a hollow fibre spiral, or other hollow fibre configurations known in the art.

Each hollow fibre within the hollow fibre group may comprise an opening for receiving the oxygenation gas. Each hollow fibre may comprise a gas-permeable wall that allows the exchange of gas between the blood and the oxygenation gas. The hollow fibre groups may be arranged to traverse (e.g. perpendicularly) the flow of blood as the blood passes through the oxygenator in order to expose the blood to the oxygenation gas present in the hollow fibre groups. The gas-blood interface may comprise a plurality of hollow fibre groups, each hollow fibre group corresponding to a respective one of the interface regions. Alternatively, the gas-blood interface may comprise a single hollow fibre group, each interface region corresponding to a portion of the single hollow fibre group.

The gas supply apparatus may comprise a gas blender configured to receive one or more supply gases and to blend the received supply gases to produce one or more oxygenation gases for supplying to the oxygenator. For example, the gas blender may be configured to receive an oxygen gas supply, a nitrogen gas supply, and/or a carbon dioxide gas supply. The oxygen gas supply may comprise a pure oxygen gas supply (i.e. the oxygen gas supply consists of 100% oxygen). The nitrogen gas supply may comprise a pure nitrogen gas supply (i.e. the nitrogen gas supply consists of 100% nitrogen). Alternatively, the nitrogen gas supply may comprise a gas mixture comprising nitrogen (e.g. the nitrogen gas supply may be air, or may comprise air). The carbon dioxide gas supply may comprise a pure carbon dioxide gas supply (i.e. the carbon dioxide gas supply consists of 100% carbon dioxide). Alternatively, the carbon dioxide gas supply may comprise a gas mixture comprising carbon dioxide (e.g. the carbon dioxide gas supply may be air, or may comprise air). The gas blender may be configured to blend the oxygen gas supply with the nitrogen gas supply and/or the carbon dioxide gas supply to produce one or more oxygenation gases. It will be noted that the blending of supply gases may produce oxygenation gases that consist solely of one of the supply gases (e.g. the gas blender may supply an oxygenation gas that consists of 100% oxygen).

It will be appreciated that the oxygenation gases may comprise additional gas components. For example, the oxygenation gas may additionally comprise one or more anaesthetic gases (e.g. isoflurane, sevoflurane, desflurane, and/or nitrous oxide). The oxygenation gases may additionally or alternatively comprise other gases common in the art (e.g. helium and/or argon).

Alternatively or additionally, the gas supply apparatus may comprise one or more valves arranged to control the flow rate and/or the composition of the first oxygenation gas and the second oxygenation gas. For example, one or more valves may be present on gas inlets that supply oxygenation gas to the first and second interface regions. Controlling these values may control the flow rate of the first and second oxygenation gases. One or more valves may be also be connected to gas supplies that supply constituent gas components of the first and second oxygenation gases. For example, one or more valves may control the flow of gas from an oxygen supply, a nitrogen supply, and/or a carbon dioxide supply. Such valves provide control of gas flow rate and composition. The gas supply apparatus may be integral with the oxygenator or may be an apparatus provided separately from the oxygenator.

Advantageously, the controller adjusts the amount of oxygen based on a measured value of a physiological parameter indicative of the oxygenation of the patient's blood to reduce the difference between the target value and the measured value. This control by the controller may be referred to as ‘closed-loop’control.

The controller may be configured to perform some or all of these operations continuously. For example, the controller may be configured to continuously receive the measured value, continuously calculate the difference, and/or continuously adjust the amount of oxygen. By ‘continuously’, it is meant that the operation may be performed in an ongoing manner with no interruptions. Alternatively or additionally, the controller may be configured to perform some or all of these operations repeatedly. For example, the controller may be configured to repeatedly receive the measured value, repeatedly calculate the difference, and/or repeatedly adjust the amount of oxygen. By ‘repeatedly’, it is meant that the operation may be performed in recurring discrete intervals (e.g. once a millisecond, once a second, once a minute).

The physiological parameter may, in general, comprise any parameter that provides a clinical indication of the level of oxygenation of a patient's blood. By ‘level of oxygenation’ it is meant a quantity that indicates how much oxygen is present in the patient's blood. The terms ‘level of oxygenation of the patient's blood’ and ‘oxygenation of the patient's blood’are used interchangeably herein.

For example, the physiological parameter may comprise the saturation of oxygen in the patient's blood and/or the partial pressure of oxygen in the patient's blood. The physiological parameter may comprise a function of the saturation of oxygen in the patient's blood and/or the partial pressure of oxygen in the patient's blood.

As used herein, the term ‘saturation of oxygen’ refers to the percentage of oxygenated haemoglobin in the patient's blood relative to the total amount of haemoglobin in the patient's blood. The terms ‘saturation of oxygen’ and ‘oxygen saturation’ are used interchangeably herein. The term ‘adjust’ refers to increasing or reducing a parameter. The saturation of oxygen may comprise the arterial saturation of oxygen, the venous saturation of oxygen, and/or the peripheral saturation of oxygen.

The arterial saturation of oxygen, abbreviated as ‘SaO2’, is defined as the saturation of oxygen in the arterial blood of a patient as measured directly from the blood. In the field of cardiac perfusion, SaO2 is considered to be the ‘true’ value of the saturation of oxygen in the arterial blood of a patient. This is in contrast to the so-called ‘peripheral’ saturation of oxygen in the arterial blood, known as SpO2. SpO2 is considered to be an estimate of SaO2 and is measured using pulse oximetry.

For example, when the measured value is a saturation of oxygen, the target saturation of oxygen may be taken as a typical saturation of oxygen in a healthy patient, such as between 95-100%, between 98-99%, or approximately 98.5% oxygen saturation. Advantageously, an oxygen saturation of 98.5% is approximately the saturation of oxygen in the blood of a healthy patient. Furthermore, choosing a saturation of oxygen that is below 100% allows ‘headroom’to increase the saturation further if required.

The controller may be configured to receive the target value of the physiological parameter. More specifically, the target value may be received by the controller as an input. For example, the controller may be configured to receive the target value as an input from a user interface. Alternatively or additionally, the controller may be pre-programmed with a target value. Alternatively or additionally, the controller may be configured to retrieve the target value from a lookup table stored locally or on a remote server.

In some embodiments, the controller is further configured to control the gas supply apparatus to decrease the amount of oxygen to which the blood is exposed by reducing the concentration of oxygen in the second oxygenation gas.

Advantageously, the controller being configured to control the gas supply apparatus to reduce the flow rate and the concentration of oxygen in the second oxygenation gas provides increased versatility of the system. In particular, both the concentration of oxygen and the flow rate of the oxygenation gases impact the delivery of oxygen and also the removal of nitrogen and carbon dioxide from the blood. Therefore, by providing a further variable (i.e. the concentration of oxygen) that can be controlled by the controller, the system is able to more accurately control both the delivery of oxygen (by controlling the concentration of oxygen) and the removal of carbon dioxide and nitrogen (by controlling the flow rate).

The controller may be configured to control the gas supply apparatus to reduce the concentration of oxygen in the second oxygenation gas before, after, and/or concurrently with the reduce of the flow rate of the second oxygenation gas. The controller may be configured to control the gas supply apparatus to reduce the concentration of oxygen in the second oxygenation gas before, after, and/or concurrently with the reduction of the concentration of oxygen in and/or the flow rate of the first oxygenation gas.

In alternative implementations, the controller may be configured to control a gas supply apparatus to, responsive to the measured value being above the target value, decrease the amount of oxygen to which the blood is exposed by reducing the concentration of oxygen in the second oxygenation gas to reduce the difference, instead of reducing the flow rate of the second oxygenation gas to reduce the difference.

In some embodiments, the controller is configured to control the gas supply apparatus to reduce the flow rate of the second oxygenation gas while maintaining a constant concentration of oxygen in and/or a constant flow rate of the first oxygenation gas.

Advantageously, the gas supply apparatus reduces the flow rate of the second oxygenation gas while concurrently maintaining a constant concentration of oxygen in and constant flow rate of the first oxygenation gas. This enables to the system to reduce oxygen delivery while maintaining a portion of the oxygenator that provides full functionality of the oxygenator. That is, despite the reduction of flow rate in the second interface region, the first interface region continues to be supplied with the first oxygenation gas that is maintained so as to continue removing undesirable gases from the blood.

In some embodiments, the constant concentration of oxygen in the first oxygenation gas is 100%.

In other words, the first oxygenation gas may consist of pure oxygen.

Advantageously, an oxygenation gas consisting solely of oxygen does not contain any nitrogen. Nitrogen is a primary component of gaseous microemboli (GME), or gas bubbles, in the blood that passes through the oxygenator. In essence, nitrogen fails to dissolve in the blood and thus leads to the formation of GME. These GME can grow in size by accumulating a layer of blood-borne materials (e.g. proteins in the blood) on their outer surface. The GME thus acts as an obstruction in the blood and can lead to serious complications, such as tissue or organ damage. The concentration of nitrogen in the oxygenation gas is too high when the partial pressure of nitrogen in the oxygenation gas is similar to, or substantially equal to, the partial pressure of nitrogen in the blood. This leads to reduced (or substantially no) removal of nitrogen from the blood when the blood passes through the oxygenator due to a lack of diffusion gradient between the nitrogen in the blood and the nitrogen in the oxygenation gas. Consequently, due to the high nitrogen content of the blood, there is also a lack of diffusion gradient between the nitrogen in the GME and the nitrogen in the blood itself. Therefore, there is little or no diffusion of nitrogen from the GME to the blood or from the blood to the oxygenation gas, and thus, there is an increased risk of GME continuing through the bloodstream, unchanged by the oxygenator. By providing an oxygenation gas of pure oxygen, a larger diffusion gradient is created between the blood and the oxygenation gas, which in turn leads to a larger diffusion gradient between the GME and the surrounding blood. Therefore, more nitrogen transfers from the blood to the oxygenation gas, and more nitrogen transfers from the GME to the blood. The removal of nitrogen is thus maximised and thus the risk of GME is considerably or completely reduced.

For similar reasons, the removal of carbon dioxide is also maximised, because the first oxygenation gas does not contain any carbon dioxide. Therefore, advantageously, the removal of undesirable gases (e.g. carbon dioxide and nitrogen) is maximised, even while the flow rate of the second oxygenation gas is reduced in order to decrease the amount of oxygen to which the blood is exposed.

In some embodiments, the controller is further configured to control the gas supply apparatus to maintain the flow rate of the second oxygenation gas at the threshold value. The controller may be further configured to increase the amount of oxygen to which the blood is exposed by increasing the concentration of oxygen in and/or the flow rate of the first oxygenation gas.

The controller may be configured to maintain the flow rate of the second oxygenation gas and increase the concentration of oxygen in and/or the flow rate of the first oxygenation gas after reducing the concentration of oxygen in and/or the flow rate of the first oxygenation gas. That is, the controller may reduce the concentration of oxygen in and/or the flow rate of the first oxygenation gas until the target value is reached, and then subsequently increase these variables. For example, this may be performed in response to a change (e.g. increase) in the target value, or in response to a change in the measured value (e.g. due to a physiological change in the patient which results in a change in metabolic rate).

The controller may generally be configured to adjust (e.g. increase and/or reduce) the concentration of oxygen in and/or the flow rate of the first oxygenation gas while maintaining the flow rate of the second oxygenation gas at the threshold value. In general, the controller may be configured to adjust the concentration of oxygen in and/or the flow rate of the first oxygenation gas and/or the second oxygenation gas in response to changes in the measured value and/or the target value of the physiological parameter. That is, the controller may be configured to maintain or adjust the concentration of oxygen in the patient's blood during a clinical intervention.

In some embodiments, the threshold value is a flow rate of zero.

That is, the controller initially reduces the flow rate of the second oxygenation gas until the flow rate reaches zero (i.e. there is no longer a flow of the second oxygenation gas in the second interface region). In this case, the controller has reduced the flow rate of the second oxygenation gas until it can be reduced no further. Thus, at this stage, the amount of oxygen to which the blood is exposed is at the minimum achievable level by controlling the flow rate of the second oxygenation gas alone. At this stage, the concentration of oxygen in the first oxygenation gas is reduced in order to continue reducing the amount of oxygen to which the blood is exposed. The threshold flow rate need not be zero, but could instead be some other value appropriate to the particular clinical situation.

Preferably, the controller may be configured to control a gas supply apparatus to: supply the first oxygenation gas to the first interface region and supply the second oxygenation gas to the second interface region, wherein the first oxygenation gas consists of pure oxygen; responsive to the measured value being above the target value (e.g. due to a drop in metabolic rate of the patient), decrease the amount of oxygen to which the blood is exposed by reducing the flow rate of the second oxygenation gas to zero in order to reduce the difference; and responsive to the flow rate of the second oxygenation gas being reduced to zero, and responsive to the measured value continuing to be above the target value, reduce the concentration of oxygen in the first oxygenation gas to reduce the difference.

In some embodiments, the controller is further configured to control the gas supply apparatus to initially, solely supply the first oxygenation gas to the first interface region without supplying the second oxygenation gas to the second interface region. The controller may be further configured to control the gas supply apparatus to adjust the amount of oxygen to which the blood is exposed by adjusting the concentration of oxygen in and/or the flow rate of the first oxygenation gas. The controller may be further configured to control the gas supply apparatus to, subsequently, initiate supply of the second oxygenation gas to the second interface region of the gas-blood interface to increase the amount of oxygen to which the blood is exposed.

It will be understood that the term ‘initially’ in this context refers to the sole supply of the first oxygenation gas occurring prior to supplying both the first oxygenation gas and the second oxygenation gas. However, it will also be understood that the controller may be configured to control the gas supply apparatus to cease supplying the second oxygenation gas, and thus the sole supply of the first oxygenation gas may also occur after the supply of both the first and second oxygenation gases.

The controller may be configured to increase and/or reduce the amount of oxygen to which the blood is exposed by increasing and/or reducing the concentration of oxygen in and/or the flow rate of the first oxygenation gas.

Advantageously, this functionality allows the blood to be exposed initially to a lower amount of oxygen by supplying only a portion of the gas-blood interface (rather than supplying the entirety of the gas-blood interface). The blood is thus exposed to oxygenation gas only in the first interface region and is thus only able to exchange gases while in the first interface region of the gas-blood interface. This is particularly advantageous when treating a patient who is currently experiencing a reduced metabolic rate (e.g. a patient in circulatory arrest) and/or a reduced saturation of oxygen (e.g. a cyanotic patient). This functionality allows, for example, the patient's current saturation of oxygen (or other physiological parameter indicative of the oxygenation of the patient's blood) to be chosen as the target saturation of oxygen (e.g. to initially match the treatment to the patient's current clinical state), even if the patient's saturation of oxygen is very low.

Initially, solely supplying the first oxygenation gas to the first interface region may comprise supplying a first oxygenation gas that consists of pure oxygen. Advantageously, supplying pure oxygen to only the first interface region (e.g. 40% of the gas-blood interface) provides a sufficiently high diffusion gradient for carbon dioxide between the first oxygenation gas and the blood passing through the oxygenator, thus ensuring adequate removal of carbon dioxide from the blood.

In particular, this functionality allows the system to expose the blood to a lower amount of oxygen (and thus operate at a lower saturation of oxygen) than if the entire gas-blood interface were to be supplied with oxygenation gas. By way of example, supplying an entire gas-blood interface with oxygenation gas that contains 20% oxygen will expose the blood to more oxygen than if only 40% of the gas-blood interface were to be supplied with oxygenation gas that contains 20% oxygen.

Advantageously, this functionality further allows the system to gradually increase the oxygenation of the patient's blood by gradually increasing the concentration of oxygen in and/or the flow rate of the first oxygenation gas. That is, the controller may be configured to increase the amount of oxygen to which the blood is exposed by adjusting the concentration of oxygen in and/or the flow rate of the first oxygenation gas. This allows the amount of oxygen to which the blood is exposed to be gradually increased to thereby increase the oxygenation of the patient's blood.

At a certain point, the system can then begin supplying a second oxygenation gas to the second interface region to further increase the amount of oxygen to which the blood is exposed and thus further increase the oxygenation of the patient's blood.

In general, the first oxygenation gas may be the same as, or different from, the second oxygenation gas. For example, the first oxygenation gas may be supplied from a first gas supply that is independent of a second gas supply that supplies the second oxygenation gas. This allows the flow rate and/or the composition of the first oxygenation gas and the second oxygenation gas to be adjusted independently. Alternatively, the first oxygenation gas and the second oxygenation gas may be supplied from a single gas supply. In this case, the controller may be configured to adjust the flow rate of the first oxygenation gas and the second oxygenation gas independently by controlling which interface regions are supplied by the single gas supply. However, in such an example, it may not be possible to alter the composition of the first oxygenation gas independently of the second oxygenation gas.

In some embodiments, the controller is configured to control the gas supply apparatus to adjust the amount of oxygen to which the blood is exposed by, responsive to the measured value being below the target value, increasing the amount of oxygen to which the blood is exposed by increasing the concentration of oxygen in and/or the flow rate of the first oxygenation gas.

In some embodiments, the controller is configured to control the gas supply apparatus to initiate the supply of the second oxygenation gas responsive to the concentration of oxygen in the first oxygenation gas having been increased to a threshold concentration. That is, the controller initially increases the concentration of oxygen in the first oxygenation gas until the concentration of oxygen reaches the threshold concentration, at which point the controller initiates the supply of the second oxygenation gas to the second interface region. For example, the threshold concentration may be 100%. In this case, the controller increases the concentration of oxygen in the first oxygenation gas until it can be increased no further. Thus, at this stage, the amount of oxygen to which the blood is exposed is at its maximum while only supplying gas to the first interface region. At this stage, the second oxygenation gas is supplied to the second interface region in order to continue increasing the amount of oxygen to which the blood is exposed. The threshold concentration need not be 100%, but could instead be some other value appropriate to a particular clinical situation.

Alternatively or additionally, the controller may be configured to control the gas supply apparatus to initiate the supply of the second oxygenation gas responsive to the flow rate of the first oxygenation gas having been increased to a threshold flow rate. For example, the threshold flow rate may be a maximum safe flow rate that can be provided through an oxygenator during extracorporeal oxygenation (such as, 5 litres per minute). The maximum safe flow rate for an oxygenation gas may be determined based on a pressure of the patient's blood in the oxygenator. For example, the maximum safe flow rate for an oxygenation gas may be chosen such that the pressure of the oxygenation gas is less than the pressure of the patient's blood in the oxygenator.

In some embodiments, the controller is further configured to, after initiating supply of the second oxygenation gas, increase the concentration of oxygen in and/or the flow rate of the second oxygenation gas to further increase the amount of oxygen to which the blood is exposed.

In some embodiments, the controller is further configured to receive a second measured value of a second physiological parameter indicative of the level of carbon dioxide in the patient's blood. The controller may be further configured to calculate a difference between the second measured value and a second target value of the second physiological parameter.

Advantageously, the controller is thereby configured to monitor the removal of carbon dioxide by the oxygenator. This is advantageous in ensuring adequate carbon dioxide removal while the controller adjusts the oxygenation gas in the oxygenator. As described above, adjusting the concentration of oxygen in and/or the flow rate of the first and second oxygenation gases impacts the removal of carbon dioxide. Therefore, by monitoring the level of carbon dioxide and comparing this to a target value, the controller can ensure that the level of carbon dioxide remains within safe limits during a clinical intervention. For example, the second physiological parameter may be partial pressure of carbon dioxide, abbreviated as ‘PaCO2’.

In some embodiments, the controller is further configured to control the gas supply apparatus to, responsive to the second measured value being above the second target value, increase the flow rate of the second oxygenation gas while concurrently reducing the concentration of oxygen in the second oxygenation gas.

Advantageously, this functionality allows the controller to respond to inadequate removal of carbon dioxide. That is, if the second measured value is above the second target value then the level of carbon dioxide is higher than desired and thus the removal of carbon dioxide is inadequate. The removal of carbon dioxide can be increased by increasing the flow rate of an oxygenation gas flowing through the oxygenator. However, by increasing the flow rate of an oxygenation gas alone, the volume of oxygen in the oxygenator will increase, leading to an increase in oxygen delivery to the patient's blood and a risk of hyperoxia. Therefore, in order to modulate oxygen delivery while increasing carbon dioxide removal, the controller is configured to reduce the concentration of oxygen in the second oxygenation gas.

This is particularly advantageous in a variety of clinical scenarios that lead to increased carbon dioxide in the patient's blood, such as during hypothermic circulatory arrest, during endovascular vein harvesting in coronary artery bypass graft procedures, or during resuscitation following a long period of inadequate perfusion (e.g. during cardiopulmonary resuscitation).

In some embodiments, the controller is configured to control the gas supply apparatus to increase the flow rate of the second oxygenation gas while concurrently reducing the concentration of oxygen in the second oxygenation gas so as to maintain a constant volume of oxygen within the second interface region.

Advantageously, the controller thereby balances the increase in flow rate by reducing the concentration of oxygen, such that the volume of oxygen in the second interface region remains constant. This will lead to an increase of carbon dioxide removal (due to the increase in flow rate) without increasing the delivery of oxygen. More specifically, maintaining a constant volume of oxygen means that the diffusion gradient between the second oxygenation gas and the patient's blood remains unchanged and thus there is little to no change in the transfer of oxygen across the gas-blood interface. Thus, the removal of carbon dioxide can be increased without having an impact on the delivery of oxygen.

It will be appreciated that these operations can be inverted to achieve the opposite physiological effect. That is, the controller may be further configured to control the gas supply apparatus to, responsive to the second measured value being below the second target value, reduce the flow rate of the second oxygenation gas while concurrently increasing the concentration of oxygen in the second oxygenation gas. The controller may be configured to reduce the flow rate of the second oxygenation gas while concurrently increasing the concentration of oxygen in the second oxygenation gas so as to maintain a constant volume of oxygen within the second interface region.

This can be particularly advantageous, for example, in cases of brain injury. A reduced level of carbon dioxide in the blood can decrease blood flow to the patient's brain, which is particularly advantageous for reducing swelling in the case of brain injury. In contrast, by reducing the flow rate of the second oxygenation gas, removal of carbon dioxide can be reduced, thereby increasing the level of carbon dioxide in the patient's blood. If desired, both the flow rate and the concentration of oxygen in the second oxygenation gas may be reduced to limit oxygenation as well as carbon dioxide removal, thereby reducing the risk of brain swelling and also the risk of brain tissue damage caused by hyperoxia. Therefore, both the flow rate and the concentration of oxygen in the second oxygenation gas may be altered to address brain swelling due to high carbon dioxide levels and also decrease the risk of brain tissue damage associated with hyperoxia.

In some embodiments, the controller is configured to control the gas supply apparatus to concurrently maintain a constant concentration of oxygen in and a constant flow rate of the first oxygenation gas while increasing the flow rate of the second oxygenation gas and concurrently reducing the concentration of oxygen in the second oxygenation gas.

The advantages of maintaining the gas flow conditions in (i.e. the concentration of oxygen in and the flow rate of) the first oxygenation gas have been discussed above. As aforementioned, this allows the controller to maintain other oxygenator functionality (e.g. carbon dioxide removal, nitrogen removal) in the first interface region.

In accordance with a second aspect, there is provided a system for control blood oxygenation in a patient, as defined in claim 15.

The system is for controlling blood oxygenation in a patient using an oxygenator having a gas-blood interface that is configured to receive blood from the patient and to expose the blood to an amount of oxygen as the blood passes through the oxygenator to produce oxygenated blood. The gas-blood interface of the oxygenator comprises a first interface region and a second interface region that are each configured to be independently supplied with a respective oxygenation gas. The system comprises a controller. The controller is configured to receive a measured value of a physiological parameter indicative of the level of carbon dioxide in the patient's blood and calculate a difference between the measured value and a target value of the physiological parameter. The controller is further configured to control a gas supply apparatus to supply the first oxygenation gas to the first interface region and supply the second oxygenation gas to the second interface region. The controller is further configured to control a gas supply apparatus to, responsive to the measured value being above the target value, increase the flow rate of the second oxygenation gas while concurrently reducing the concentration of oxygen in the second oxygenation gas.

It will be understood that the process of blood oxygenation (as compared to the parameter of oxygenation) involves the addition and/or removal of multiple blood gases (e.g. oxygen, carbon dioxide, and nitrogen), and does not merely refer to the addition of oxygen to the blood. In this regard, the system may be regarded as a system for controlling blood carbon dioxide levels in a patient using an oxygenator.

Advantageously, the controller is thereby configured to monitor the removal of carbon dioxide by the oxygenator, as discussed above. Advantageously, this functionality allows the controller to respond to inadequate removal of carbon dioxide, as discussed above.

In some embodiments, the controller is configured to control the gas supply apparatus to increase the flow rate of the second oxygenation gas while concurrently reducing the concentration of oxygen in the second oxygenation gas so as to maintain a constant volume of oxygen within the second interface region.

In some embodiments, the controller is configured to control the gas supply apparatus to concurrently maintain a constant flow rate and/or a constant concentration of the first oxygenation gas while increasing the flow rate of the second oxygenation gas and concurrently reducing the concentration of oxygen in the second oxygenation gas.

In some embodiments, the system further comprises the gas supply apparatus. The gas supply apparatus may be configured to control the flow rate of and/or the concentration of oxygen in the first oxygenation gas and the second oxygenation gas.

### In Some Embodiments, the System Further Comprises the Oxygenator.

The system may be supplied independently of the oxygenator and/or the gas supply apparatus. For example, the system may comprise a control module or control unit that is configured for connection to the oxygenator and/or the gas supply apparatus. The system may comprise an oxygenator and/or a gas supply apparatus that are already connected to and/or integral with the controller.

While the systems described herein generally comprise a single oxygenator, in some implementations, the system may comprise a plurality of oxygenators. The oxygenators may be arranged in series, such that blood received from the patient passes through a first oxygenator, followed by a second oxygenator, and so on. Alternatively, the oxygenators may be arranged in parallel and/or in any combination of in series and in parallel. In such an example, each oxygenator in the system may be connected to a separate, respective, gas supply apparatus and/or to separate, respective, independent gas supplies. That is, each oxygenator may be configured to expose the blood to one or more oxygenation gases that are controllable independently of the one or more oxygenation gases in another oxygenator in the system. Any or all of the oxygenators in the system may comprise the features and characteristics of the oxygenator described herein. For example, one or more (e.g. all) of the oxygenators in the system may comprise a gas-blood interface that is divided into a plurality of interface regions. For example, the system may comprise a plurality of oxygenators, each oxygenator comprising a gas-blood interface that comprises a first interface region and a second interface region. Alternatively, a single oxygenator in the system may comprise multiple interface regions, and any additional oxygenators in the system may comprise a gas-blood interface with a single interface region (i.e. an undivided gas-blood interface). In this respect, it will be understood that the controller described herein may be configured to control a system comprising a plurality of oxygenators.

In some embodiments, the oxygenator comprises a gas inlet zone for receiving oxygenation gas into the gas-blood interface. The gas inlet zone may comprise a partition dividing the gas inlet zone into a first gas inlet region and a second gas inlet region. The first gas inlet region may be configured to receive the first oxygenation gas. The first gas inlet region may be configured to provide the first oxygenation gas to the first interface region. The second gas inlet region may be configured to receive the second oxygenation gas. The second gas inlet region may be configured to provide the second oxygenation gas to the second interface region.

The gas-blood interface of the oxygenator may comprise a plurality of hollow fibre groups, each hollow fibre group corresponding to one of the interface regions, and thus to one of the gas inlet regions. Alternatively, the gas-blood interface may comprise a single hollow fibre group, and each interface region (and thus each gas inlet zone) may correspond to a portion of the single hollow fibre group.

The one or more partitions may separate the gas-blood interface. For example, a partition may extend from the gas inlet zone and into the gas-blood interface, and may extend through the gas-blood interface. Where the gas-blood interface comprises a plurality of hollow fibre groups, the hollow fibre groups may be separated from one another by a gap in which a partition is located.

At least one of the one or more partitions may be movable. For example, the one or more partitions may be movable to adjust the relative sizes of each of the gas inlet regions. The controller may be configured to adjust the positions of the one or more partitions (e.g. by activating a motor coupled to the one or more partitions). For example, the controller may be configured to receive an input representative of a desired value of the physiological parameter and/or a desired position of the one or more partitions. The controller may further be configured to adjust the positions of the one or more partitions responsive to the input. The controller may further be configured to convert a desired value of the physiological parameter into a position of the one or more partitions. For example, by adjusting the one or more partitions to increase the size of an interface region that is supplied with oxygenation gas, the exposure of the blood to the oxygenation gas increases which, in turn, may increase the level of oxygenation in the blood.

In some embodiments, the system further comprises a sensor configured to measure the physiological parameter. The controller may be configured to receive the measured value from the sensor.

The sensor may be configured to measure the physiological parameter (e.g. saturation of oxygen present in the oxygenated blood) via spectrophotometry performed on the blood line. Advantageously, spectrophotometry provides an accurate means of measuring physiological parameters relating to blood oxygenation. Furthermore, spectrophotometry is a non-invasive method that may be performed during surgery without contacting the patient's blood (e.g. to measure pH or temperature).

Spectrophotometry for measuring saturation of oxygen involves a white light source being shone towards the blood in the blood line. The white light is at least partially reflected by the blood and the reflected light is detected by a receiver. The receiver will only detect wavelengths that are reflected by the blood, and will not detect wavelengths that are absorbed by the blood. Some wavelengths in the white light are absorbed more strongly by oxygenated haemoglobin and others are absorbed more strongly by de-oxygenated haemoglobin. By measuring the absorption of each wavelength and comparing the relative absorptions, the proportion of oxygenated haemoglobin to de-oxygenated haemoglobin can be determined, which can, in turn, be used to determine the saturation of oxygen in the blood.

To assist in the spectrophotometry, the blood line may be transparent. For example, the blood line may be transparent to each of the wavelengths of light used in the spectrophotometry.

In some embodiments, the sensor is positioned on a blood line. The blood line may be connected to the oxygenator. The blood line may be configured to carry the oxygenated blood from the oxygenator to the patient.

Advantageously, the sensor measures the physiological parameter following oxygenation by the oxygenator. The sensor may be considered to be ‘positioned downstream of the oxygenator’. That is, the blood flows past the sensor after having passed through the oxygenator. In other words, the sensor is positioned downstream with respect to the direction of blood flow. In this regard, the physiological parameter measured by the sensor may be considered to constitute an ‘arterial’ measurement. This is in contrast to a ‘venous’ measurement, which would be taken upstream of the oxygenator (for example, immediately after the blood exits the patient, or between a venous reservoir and the oxygenator).

Venous measurements of physiological parameters are heavily influenced by physiological factors of the patient. For example, venous oxygen saturation and venous partial pressure are functions of the patient's metabolic rate, blood flow, haemoglobin levels, and other physiological parameters. Performing venous measurements therefore inherently incorporates uncertainty and a lack of accuracy into any subsequent control of the patient's blood oxygen level based thereon. A clinician would need an understanding of anaesthetic factors, the degree of paralysis of a patient, the patient's temperature, the state of a patient's capillary beds, autonomic responses (e.g. immune or inflammatory reactions), and a variety of other factors in order to control oxygenation based on venous measurements. Therefore, using a sensor to measure the physiological parameter following oxygenation provides increased accuracy and safety of the system for controlling blood oxygenation.

In some embodiments, the system further comprises a venous reservoir configured to receive the blood from the patient. The system may further comprise a pump configured to drive blood flow from the venous reservoir through the oxygenator.

The venous reservoir may be positioned upstream of the oxygenator. The venous reservoir may be configured to be positioned between the oxygenator and the patient. That is, the venous reservoir may receive deoxygenated blood from the patient. The pump may be configured to draw deoxygenated blood from the venous reservoir, and to cause the deoxygenated blood to flow into the oxygenator. The pump may be positioned upstream of the oxygenator. The pump may be positioned downstream of the venous reservoir. The pump may be positioned between the venous reservoir and the oxygenator. The pump may be a centrifugal pump or a roller (peristaltic) pump.

It will be understood that any features, functions, characteristics, or advantages described with respect to the first aspect may be applied to the second aspect, and vice versa. Similarly, the features, functions, characteristics, or advantages described with respect to a system may be applied to a corresponding method, and vice versa.

Example embodiments will now be described with reference to the accompanying drawings in which:

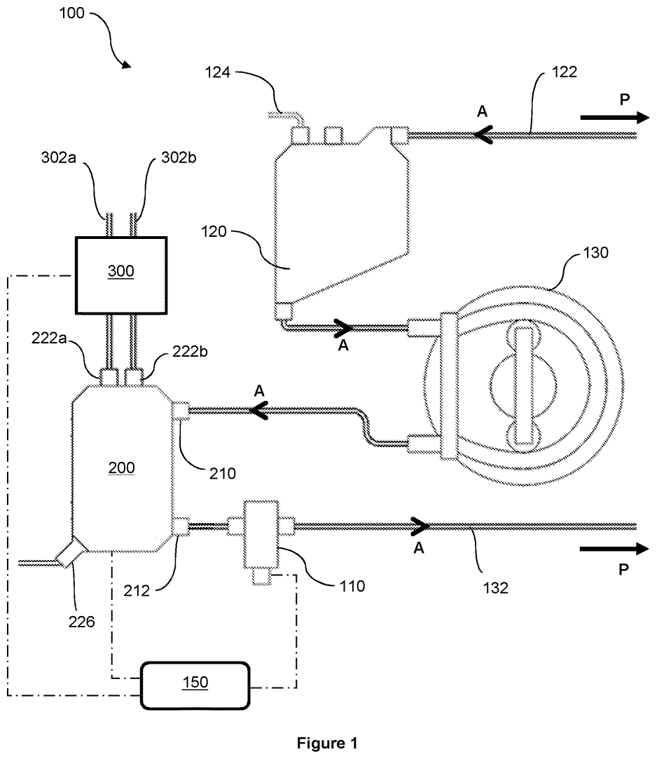

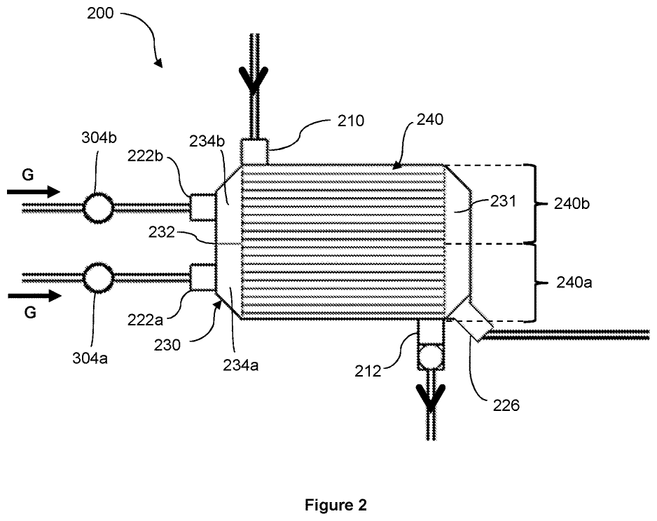

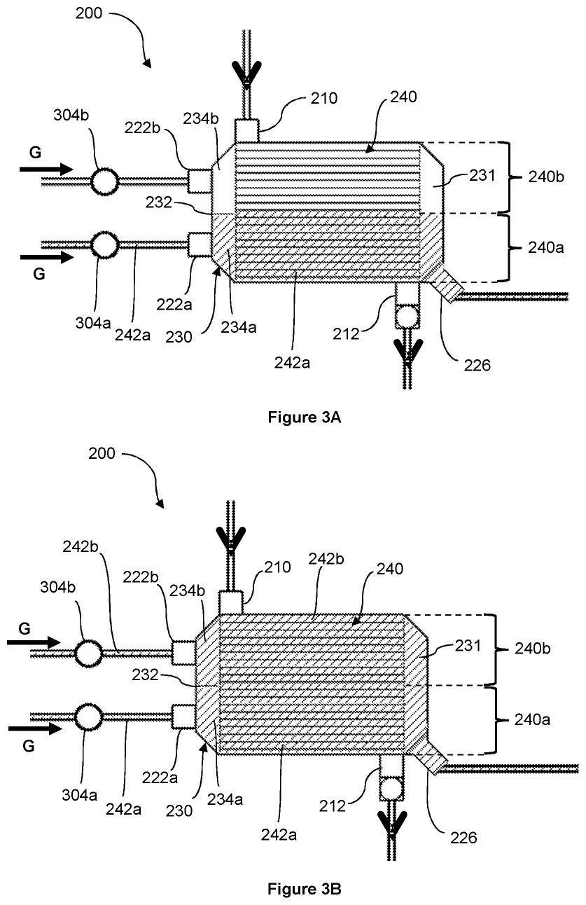

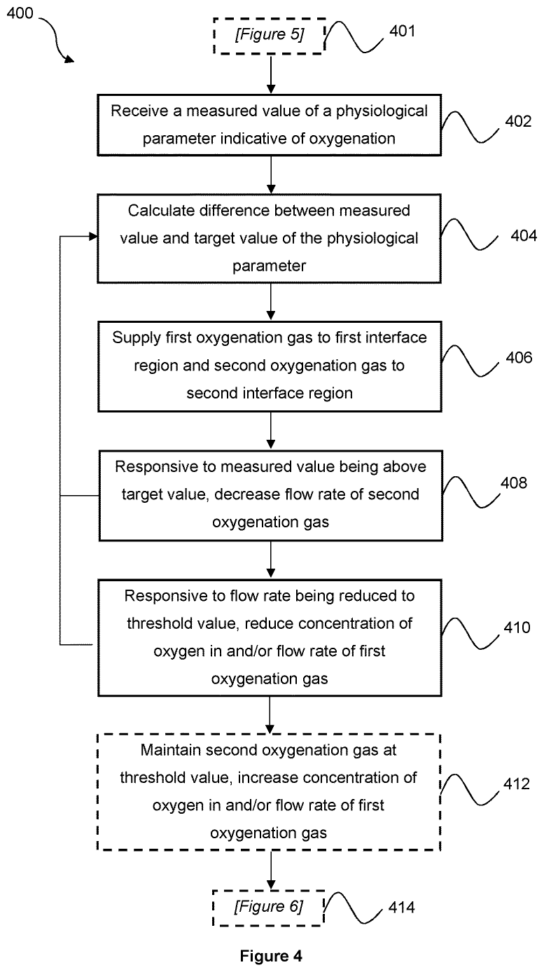

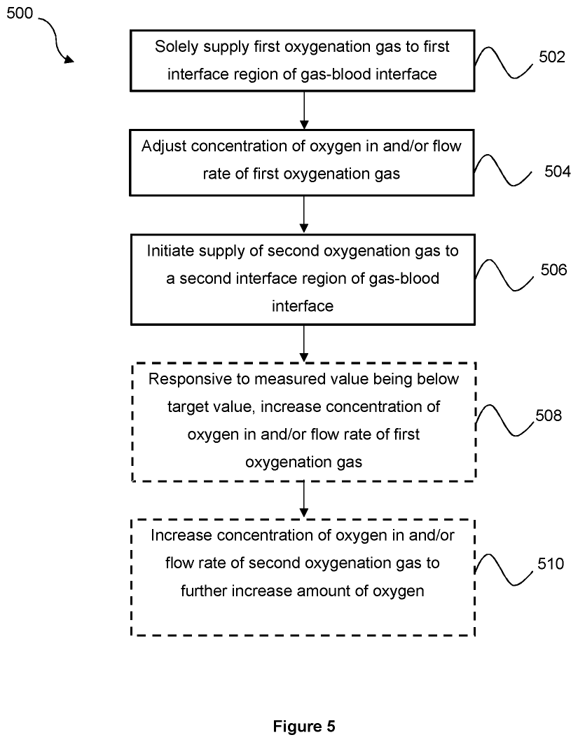

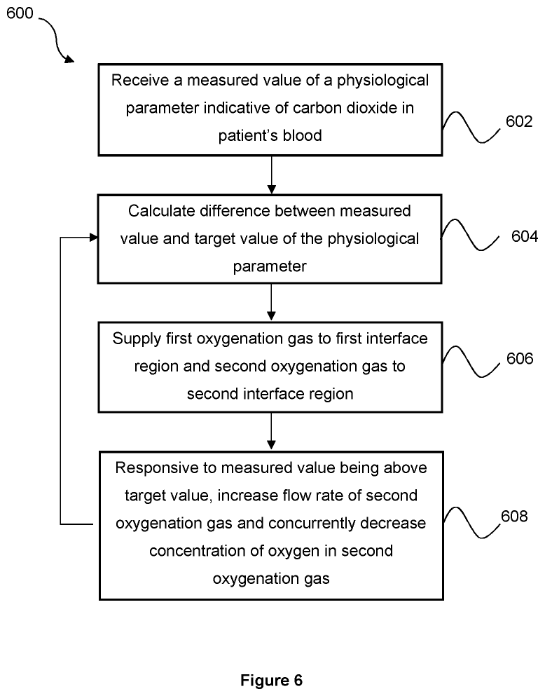

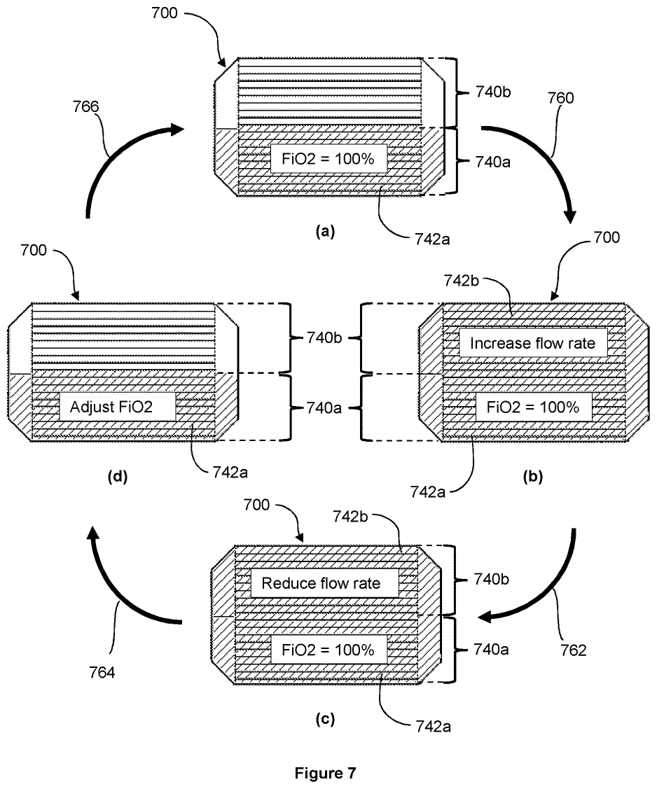

FIG. 1 is a schematic diagram of a system for controlling blood oxygenation in a patient;
FIG. 2 is a schematic diagram of an example of an oxygenator for a system for controlling blood oxygenation in a patient;
FIG. 3A is a schematic diagram of the oxygenator of FIG. 2 having a first interface region supplied with a first oxygenation gas;
FIG. 3B is a schematic diagram of the oxygenator of FIG. 2 having a first interface region supplied with a first oxygenation gas and a second interface region supplied with a second oxygenation gas;
FIG. 4 is a flowchart of a first example of a method of controlling blood oxygenation in a patient using an oxygenator;
FIG. 5 is a flowchart of a second example of a method of controlling blood oxygenation in a patient using an oxygenator;
FIG. 6 is a flowchart of a third example of a method of controlling blood oxygenation in a patient using an oxygenator;
FIG. 7 is a schematic diagram illustrating states of operation of the oxygenator of FIG. 2.

FIG. 1 depicts a system 100 for controlling blood oxygenation in a patient. The position of the patient relative to the system 100 is illustrated by arrows P, which indicate which blood lines (see below) lead to the patient. The system 100 comprises an oxygenator 200. The oxygenator 200 comprises a gas-blood interface 240 (see FIG. 2) that is configured to receive blood from the patient and to expose the blood to an amount of oxygen as the blood passes through the oxygenator 200 to produce oxygenated blood. The structure of the oxygenator 200 will be described in greater detail with reference to FIG. 2.

The system 100 further comprises a gas supply apparatus 300. The gas supply apparatus 300 is configured to control the flow rate of and/or the concentration of oxygen in a first oxygenation gas 242a and a second oxygenation gas 242b (shown in FIGS. 3A and 3B). The first oxygenation gas 242a may be supplied to the oxygenator 200 via a first gas inlet 222a. The second oxygenation gas 242b may be supplied to the oxygenator 200 via a second gas inlet 222b.
The gas supply apparatus 300 is depicted as a single component that receives a first gas supply 302a and a second gas supply 302b. For example, the gas supply apparatus 300 may comprise a gas blender configured to receive one or more supply gases and blend the supply gases to produce one or more oxygenation gases for supplying to the oxygenator 200. It will, however, be appreciated that the gas supply apparatus 300 may comprise a plurality of components. For example, in addition to or instead of the gas blender, the gas supply apparatus 300 may comprise one or more valves that control the flow rate of one or more supply gases to allow the gas supply apparatus 300 to control the flow rate of and/or the concentration of oxygen in the one or more oxygenation gases supplied to the oxygenator 200. The gas supply apparatus 300 may comprise valves, actuators and/or other gas flow control mechanisms that control the gas flow into and/or out of the oxygenator 200.

The system 100 further comprises a sensor 110. The sensor 110 is positioned downstream of the oxygenator 200. In the depicted example, the sensor 110 is positioned upstream of the patient on a blood line (in this case, arterial line 132) that leaves the oxygenator 200. The sensor 110 may be configured to measure a physiological parameter indicative of the oxygenation of the patient's blood. For example, the sensor 110 may be configured to measure a saturation of oxygen present in the oxygenated blood (i.e. in the blood following oxygenation by the oxygenator 200). For example, the sensor 110 may be configured to measure the saturation of oxygen present in the oxygenated blood via spectrophotometry performed on the arterial line 132. In this case, the sensor 110 is considered to measure the ‘arterial saturation of oxygen’ (SaO2). It will be appreciated that other technologies for measuring the saturation of oxygen may be used in sensor 110. In addition, the sensor 110 may be positioned downstream of the oxygenator 200 but not upstream of the patient. For example, the sensor 110 may comprise a pulse oximeter that is attached to the patient. In this case, the sensor 110 would be considered to measure the ‘peripheral saturation of oxygen’ (SpO2). SpO2 is understood in the art to represent an estimate of SaO2, which is considered to be the ‘true’ value of the saturation of oxygen in the arterial blood of the patient.

Alternatively or additionally, the sensor 110 may be configured to measure a physiological parameter indicative of the level of carbon dioxide in the patient's blood/For example, the sensor 110 may be configured to measure a partial pressure of carbon dioxide present in the oxygenated blood (i.e. in the blood following oxygenation by the oxygenator 200). For example, the sensor 110 may be configured to measure the partial pressure of carbon dioxide via blood gas analysis, such as an arterial blood gas (ABG) test. In such a case, the sensor 110 may be an invasive blood gas sensor. It will be appreciated that other technologies for measuring the partial pressure of carbon dioxide may be used in sensor 110. The system 100 may comprise an additional sensor (not shown) to measure the partial pressure of carbon dioxide. That is, the system may comprise a first sensor 110 for measuring the physiological parameter indicative of the oxygenation of the patient's blood and a second sensor (not shown) for measuring the physiological parameter indicative of the level of carbon dioxide in the patient's blood.

The system 100 further comprises a controller 150. The controller 150 is configured to receive the measured value from the sensor 110 (or any other sensor present in system 100). The controller 150 is further configured to calculate a difference between the measured value and a target value of the physiological parameter. The functionality of the controller 150 will be described in greater detail below, with reference to FIGS. 3A to 5.

The controller 150 is communicatively connected to the oxygenator 200, the gas supply apparatus 300, and the sensor 110, as depicted by the dash-dot lines in FIG. 1. That is, controller 150 may be configured to communicate with the oxygenator 200, the gas supply apparatus 300, and the sensor 110. This communication may occur via hardware connections (e.g. wired connections) or via wireless communication. In this regard, it will be understood that the dash-dot lines depicted in FIG. 1 are merely illustrative and do not necessarily represent physical connections between components. Furthermore, it will be understood that the controller 150 may be communicatively connected to more or fewer components in system 100. For example, controller 150 may not necessarily communicate directly with the oxygenator 200, but may instead be configured to communicate with only the gas supply apparatus 300. Similarly, the controller 150 may not necessarily communicate directly with the sensor 110, but may instead be configured to communicate with an intermediate transceiver that relays measurements from the sensor 110 to the controller 150. The controller 150 may further be configured to communicate with the pump 130 and/or other components present in system 100. Other arrangements of communicative connections between components will be readily understood by the skilled person.

It is noted that ‘communication’ with the controller 150 refers to both the reception of data (e.g. measurements) by the controller 150 and the transmission of data (e.g. instructions or commands) by the controller 150.

The controller 150 may comprise any suitable type of data processing device, such as a microprocessor, a microcontroller or an application specific integrated circuit (ASIC). The data processing device may be communicatively coupled to a memory (e.g., a volatile memory, a non-volatile memory, or both volatile and non-volatile memories), upon which is stored processor-executable instructions that cause the controller to perform any of the methods disclosed herein.

As shown in FIG. 1, the system 100 is configured to receive blood from the patient via a venous line 122 and to return blood from the patient via an arterial line 132. The connection of the system 100 to the patient is illustrated by arrows P in FIG. 1. The system 100 further comprises a venous reservoir 120 that is configured to receive blood from the patient via the venous line 122. The venous reservoir 120 may additionally be configured to receive blood from the patient via one or more salvage lines, one or more purge lines, and/or one or more lines configured to carry surgical fluids (e.g. priming solutions, volume expanders, blood, and/or drugs), as represented by line 124 in FIG. 1. The venous reservoir 120 is positioned upstream of the oxygenator 200, between the patient and the oxygenator 200. It will be appreciated that, in other implementations, the exact arrangement of the venous reservoir 120 and the pump 130 may vary. In fact, the system 100 may not necessarily comprise the venous reservoir 120 and the pump 130. In such a case, the oxygenator 200 may be configured to receive the blood directly from the patient.

The blood, once collected in the venous reservoir 120, is driven through the oxygenator 200 by a pump 130. The pump 130 is located downstream of the venous reservoir 120 and upstream of the oxygenator 200. In the depicted embodiment, pump 130 is a roller (or peristaltic) pump. However, it will be appreciated that, depending on the circumstance, other types of pump may be used, such as a centrifugal pump. The blood exits the pump 130 and is received by the oxygenator 200 via a blood inlet 210. The blood is oxygenated by the oxygenator 200 and then exits the oxygenator via a blood outlet 212, before passing the sensor 110 and being returned to the patient via the arterial line 132. The blood flow path through the system 100 is shown by arrows A in FIG. 1.

While the system 100 is depicted to comprise various components (e.g. the sensor 110, the oxygenator 200, the gas supply apparatus 300, the venous reservoir 120, the pump 130), it will be appreciated that the system 100 may simply comprise the controller 150. That is, it is not essential that the system 100 comprises the sensor 110, the oxygenator 200, or any of the other components depicted in FIG. 1. The system 100 may instead be provided as a controller 150 that is configured to communicate with and/or control any of the aforementioned components. The additional components in FIG. 1 are merely included within the system 100 for illustrative purposes in order to show the context in which the controller 150 may operate. However, the system 100 may comprise any or all of the additional components in FIG. 1.

FIG. 2 depicts an example structure of the oxygenator 200. As described above, the oxygenator 200 comprises a blood inlet 210 for receiving blood from the patient and a blood outlet 212 for returning blood to the patient. The oxygenator 200 further comprises two gas inlets for receiving two oxygenation gases into the oxygenator 200, as depicted by arrows G. Oxygenator 200 comprises a first gas inlet 222a and a second gas inlet 222b. The first gas inlet 222a and the second gas inlet 222b are each fluidly connected to a gas inlet zone 230. The oxygenator 200 further comprises a gas outlet 226 (which may be referred to as a gas exhaust 226) for releasing waste gas from the oxygenator 200. The oxygenation gas exits the oxygenator 200 from the gas outlet 226 via a gas outlet zone 231.

Gas inlet zone 230 comprises a partition 232 that divides the gas inlet zone 230 into a plurality of (in this case, two) gas inlet regions 234a, 234b. Each gas inlet region 234a, 234b is configured to receive a different one of the respective oxygenation gases. More specifically, a first gas inlet region 234a is configured to receive oxygenation gas from the first gas inlet 222a and the second gas inlet region 234b is configured to receive oxygenation gas from the second gas inlet 222b.
The oxygenator 200 further comprises a gas-blood interface 240. The gas inlet zone 230 is fluidly connected with the gas-blood interface 240. The gas outlet zone 231 is also fluidly connected with the gas-blood interface 240. That is, the oxygenation gases enter the gas-blood interface 240 from the gas inlet zone 230 and exit the gas-blood interface 240 via the gas outlet zone 231.

The gas-blood interface 240 may comprise one or more hollow fibre groups, each hollow fibre group comprising a plurality of hollow fibres. Each hollow fibre group comprises inlet potting in fluid connection with the gas inlet zone 230. Each hollow fibre group comprises outlet potting in fluid connection with the gas outlet zone 231.

The gas-blood interface 240 is configured to be supplied with oxygenation gas to expose the blood to an amount of oxygen. For example, one or more oxygenation gases may enter the hollow fibre groups via the inlet potting from the gas inlet zone 230. The blood enters the oxygenator 200 via the blood inlet 210. The blood and the one or more oxygenation gases pass through the gas-blood interface 240 as they pass through the oxygenator 200. The gas-blood interface 240 is configured to permit gaseous exchange between the blood and the oxygenation gases supplied to the gas-blood interface 240. This includes the transfer of gases (e.g. oxygen, carbon dioxide, nitrogen) out of the blood and into the oxygenation gas as well as the transfer of gases (e.g. oxygen, carbon dioxide) into the blood and out of the oxygenation gas. Following gaseous exchange, the blood (now oxygenated) exits the oxygenator 200, towards the patient, via blood outlet 212 and the oxygenation gas (now waste gas) exits the oxygenator 200 via gas outlet 226.

As can be seen in FIG. 2, the presence of multiple gas inlet regions 234a, 234b effectively allows different proportions of the gas-blood interface 240 to be supplied with respective oxygenation gases. In this regard, the gas-blood interface 240 comprises a plurality of (in this case, two) interface regions 240a, 240b that are each configured to be independently supplied with a respective oxygenation gas.

The partition 232 as shown in FIG. 2 divides the gas inlet zone 230 into the first gas inlet region 234a and the second gas inlet region 234b that are of equal size. That is, the partition 232 divides the gas inlet zone 230 (and thus the gas-blood interface 240) in half, such that each of the first gas inlet region 234a and the second gas inlet region 234b comprises 50% of the gas inlet zone 230. In turn, the partition 232 thus acts to divide the gas-blood interface 240 in half such that each of the first interface region 240a and the second interface region 240b comprises 50% of the gas-blood interface 240. However, it will be appreciated that this divide is merely an example and the partition 232 (and/or multiple partitions) may be located at various positions in order to create different splits of the gas inlet zone 230 and of the gas-blood interface 240. For example, the partition 232 may be positioned such that the first gas inlet region 234a comprises 40% of the gas inlet zone 230 and the second gas inlet region 234b comprises 60% of the gas inlet zone 230. That is, the gas-blood interface 240 may be divided such that the first interface region 240a comprises 40% of the gas-blood interface 240 and the second interface region 240b comprises 60% of the gas-blood interface 240.

The partition 232 as shown in FIG. 2 extends through the gas inlet zone 230, but does not extend through the gas-blood interface 240. In such a case, the partition 232 may abut the inlet potting of the hollow fibre group to prevent gas flow between the interface regions 240a, 240b. Alternatively, the partition 232 may not abut the inlet potting because leakage of gas flow between the gas inlet regions 234a, 234b may be permissible. In other examples, the partition 232 may extend through the gas inlet zone 230 and at least partially through the gas-blood interface 240 to physically separate the gas-blood interface 240 into interface regions 240a, 240b.

It will be noted that, in general, the oxygenation gases supplied to each of the gas inlet regions 234a, 234b (and thus to each of the interface regions 240a, 240b) may originate from the same or different gas supplies. The oxygenation gases may have the same composition or different compositions.

As described above, the gas supply apparatus 300 may comprise one or more valves arranged to control the flow rate and/or the composition of the first oxygenation gas and the second oxygenation gas. For example, as depicted in FIG. 2, the gas supply apparatus may comprise a first valve 304a configured to control the flow of oxygenation gas into the first gas inlet 222a and a second valve 304b configured to control the flow of oxygenation gas into the second gas inlet 222b. The controller 150 may be configured to control (e.g. open or close) the first valve 304a and the second valve 304b independently of each other to adjust (e.g. increase or reduce) the flow rate through each of the interface regions 240a, 240b. Alternatively or additionally, the gas supply apparatus 300 may comprise a gas blender for controlling the gas flow and/or composition, as described above. It will be appreciated that other examples of valves, actuators, blenders, gas flow controllers, or other components for controlling the flow rate and/or the composition of gases may be used instead of and/or in addition to the valves or gas blender described herein.

Turning to FIGS. 3A and 3B, the general functionality of the oxygenator 200 (and the system 100) will be described. As already described, the partition 232 allows the gas inlet regions 234a, 234b to be independently supplied with respective oxygenation gases. Starting with FIG. 3A, the first gas inlet 222a may be supplied with a first oxygenation gas 242a. The first gas inlet 222a may be supplied with the first oxygenation gas 242a without supplying oxygenation gas to the second gas inlet 222b (i.e. oxygenation gas is solely supplied to the first gas inlet 222a). This supplies the first oxygenation gas 242a to the first gas inlet region 234a and thus to the first interface region 240a.

In this situation, the blood passing through the oxygenator 200 is exposed to the first oxygenation gas 242a through only a portion (in this case, 50%) of the gas-blood interface 240. That is, while the blood passes through the second interface region 240b, the blood is exposed to substantially no oxygenation gas. The blood is only exposed to the first oxygenation gas 242a when the blood passes through the first interface region 240a. Therefore, the amount of oxygen to which the blood is exposed in the oxygenator 200 can be kept low. The concentration and/or the flow rate of the first oxygenation gas 242a can be adjusted as described above to further adjust (e.g. increase and/or reduce) the amount of oxygen to which the blood is exposed. It will be appreciated that, depending on the relative sizes of the first interface region 240a and the second interface region 240b, a similar effect may be achieved by supplying oxygenation gas only to the second interface region 240b via the second gas inlet 222b.

Moving to FIG. 3B, the first oxygenation gas 242a is supplied to the first gas inlet 222a as in FIG. 3A and also a second oxygenation gas 242b is supplied to the second gas inlet 222b. This supplies the second oxygenation gas 242b to the second gas inlet region 234b and thus to the second interface region 240b.

In this situation, the blood passing through the oxygenator 200 is exposed to the second oxygenation gas 242b when the blood passes through the second interface region 240b, and is subsequently exposed to the first oxygenation gas 242a when the blood passes through the first interface region 240a. Therefore, the amount of oxygen to which the blood is exposed in the oxygenator 200 is increased relative to the situation depicted in FIG. 3A. The concentration and/or the flow rate of the first oxygenation gas 242a and/or the second oxygenation gas 242b can be adjusted as described above to further adjust (e.g. increase and/or reduce) the amount of oxygen to which the blood is exposed. It will be appreciated that the flow rate and/or the composition of the first oxygenation gas 242a can be adjusted independently of the flow rate and/or the composition of the second oxygenation gas 242b.

In this way, the controller 150 can use the oxygenator 200 and the gas supply apparatus 300 to control the amount of oxygen to which the blood is exposed in the oxygenator 200.

The first oxygenation gas 242a and/or the second oxygenation gas 242b may consist of pure oxygen. The first oxygenation gas 242a and/or the second oxygenation gas 242bmay comprise a mixture of oxygen and nitrogen.

While a specific example of an oxygenator that may be used with the system 100 has been described, it will be appreciated that other oxygenators may be used that achieve the same or similar functionality. For example, an oxygenator may comprise multiple gas inlet zones that each connect to a separate gas inlet. Alternatively, the gas inlet(s) may connect directly to the gas-blood interface, which may be separated into a plurality of interface regions within the oxygenator. It will be appreciated that other structures of oxygenator exist that allow for a plurality of interface regions to be independently supplied with a respective oxygenation gas. Furthermore, while oxygenators have been shown with two gas inlet regions and two interface regions, it will be appreciated that substantially any number of gas inlet regions and interface regions may be present. The more gas inlet regions and interface regions that are present, the more independent oxygenation gases that can be supplied to the gas-blood interface.

The functionality of the system 100 as a whole will now be described with reference to FIGS. 4 to 7. It will be appreciated that the controller 150 may be configured to control the gas supply apparatus 300 to perform any of the methods described herein.

FIG. 4 depicts a first example method 400 of controlling blood oxygenation in a patient, as contemplated herein. The controller 150 may be configured to perform any or all of the operations of the method 400. The method 400 comprises receiving 402 a measured value of a physiological parameter indicative of the oxygenation of the patient's blood. The measured value may, for example, be measured by the sensor 110 and received by the controller 150 from the sensor 110. The method 400 further comprises calculating 404 a difference between the measured value and a target value of the physiological parameter. The method 400 further comprises supplying 406 the first oxygenation gas 242ato the first interface region 240a of the gas-blood interface 240 and the second oxygenation gas 242bto the second interface region 240b of the gas-blood interface 240. The method 400 further comprises, responsive to the measured value being above the target value, decreasing the amount of oxygen to which the blood is exposed by reducing 408 the flow rate of the second oxygenation gas 242 to reduce the difference. The method 400 further comprises, responsive to the flow rate of the second oxygenation gas 242b being reduced to a threshold value (and the measured value of the physiological parameter still being above the target value), reducing 410 the concentration of oxygen in and/or the flow rate of the first oxygenation gas 242a. Following reducing 408 the flow rate of the second oxygenation gas 242b and/or following reducing 410 the concentration of oxygen in and/or the flow rate of the first oxygenation gas 242a, the method may comprise recalculating the difference between the measured value and the target value, as indicated by the return arrows in FIG. 4. It will be appreciated that various operations within the method 400, and the method 400 as a whole, may be performed repeatedly and/or continuously. For example, the method 400 may comprise receiving updated measured values and repeating the method 400 in response to the updated measured value.

It will be appreciated that the supplying of the first and second oxygenation gases may occur at a different point within the method 400. For example, the first and second oxygenation gases may be supplied before a measured value is received.

Advantageously, the method 400 is particularly beneficial in the treatment of a patient experiencing low metabolic rate. The patient's metabolic rate may drop during surgery involving extracorporeal oxygenation. For example, the patient's metabolic rate may be deliberately reduced by a clinician by reducing the temperature of the patient. Following the reduction in metabolic rate, the patient's body will consume less oxygen from the blood and thus the physiological parameter indicative of the oxygenation of the blood will increase. Following the increase, the controller 150, being configured to perform method 400, responds by reducing the flow rate of the second oxygenation gas 242b in the second interface region 240b. A constant concentration and/or flow rate of the first oxygenation gas 242a may be maintained while the flow rate of the second oxygenation gas 242b is reduced.

Advantageously, the continued supply of the first oxygenation gas 242a allows the system 100 to continue to provide a ‘sweep’ functionality. That is, the first interface region 240a still provides adequate removal of carbon dioxide and nitrogen, as well as a base level of oxygenation, while the flow rate in the second interface region 240b is reduced in order to reduce the delivery of oxygen. Only once the flow rate of the second oxygenation gas 242b has been reduced to a threshold value (e.g. zero) does the controller 150 then reduce the concentration of oxygen in and/or the flow rate of the first oxygenation gas 242a. This allows the amount of oxygen to which the blood is exposed to be further reduced to reach the target value.

Optionally, the method 400 may further comprise maintaining the flow rate of the second oxygenation gas 242b at the threshold value and increasing the amount of oxygen to which the blood is exposed by increasing 412 the concentration of oxygen in and/or the flow rate of the first oxygenation gas 242a. For example, the concentration of oxygen in and/or the flow rate of the first oxygenation gas 242a may be increased back to their original levels (i.e. their levels prior to being reduced at operation 410 responsive to the reduction of the flow rate of the second oxygenation gas 242b to the threshold value). For example, the concentration of oxygen in the first oxygenation gas 242a may be increased to 100%.

As indicated by feature 401, the method 400 may be preceded by method 500, described below with reference to FIG. 5. As indicated by feature 414, the method 400 may be followed by method 600, described below with reference to FIG. 6. Alternatively, the method 600 may be performed independently of method 400.

FIG. 5 depicts a second example method 500 of controlling blood oxygenation in a patient, as contemplated herein. The controller 150 may be configured to perform any or all of the operations of the method 500. The method 500 comprises, initially, solely supplying 502 the first oxygenation gas 242a to the first interface region 240a of the gas-blood interface 240 without supplying the second oxygenation gas 242bto the second interface region 240b. The method 500 may further comprise adjusting the amount of oxygen to which the blood is exposed by adjusting 504 the concentration of oxygen in and/or the flow rate of the first oxygenation gas 242a. The method 500 further comprises, subsequently, initiating 506 supply of the second oxygenation gas 242b to the second interface region 240b of the gas-blood interface 240 to increase the amount of oxygen to which the blood is exposed. Initiating 506 the supply of the second oxygenation gas 242b may be responsive to the concentration of oxygen in the first oxygenation gas 242a having been increased to a threshold concentration (e.g. 100% oxygen).

In an example scenario, oxygenation gas (in the form of the first oxygenation gas 242a) may initially be supplied solely to the first interface region 242a. At this stage, the first oxygenation gas 242a may comprise a mixture of nitrogen and oxygen. In this regard, the blood is exposed to a ‘low’ amount of oxygen (and thus a ‘low’ saturation of oxygen in the patient's blood can be achieved). The concentration of oxygen in the first oxygenation gas 242a may be gradually increased to increase the amount of oxygen to which the blood is exposed (and thus increase the saturation of oxygen in the blood). Once the concentration of oxygen in the first oxygenation gas 242a reaches 100%, it is no longer possible to increase the amount of oxygen to which the blood is exposed by increasing the concentration of oxygen in the first oxygenation gas 242a. Therefore, supply of the second oxygenation gas 242b is initiated to further increase the amount of oxygen to which the blood is exposed. The concentration of oxygen in and/or the flow rate of the second oxygenation gas 242b can then be further adjusted to adjust the amount of oxygen to which the blood is exposed.

Advantageously, the method 500 is particularly beneficial in the treatment of a patient experiencing low oxygen saturation. Due to the current low saturation of oxygen in the patient's blood, the oxygenator 200 should be operated to expose the blood to a smaller amount of oxygen so as to reduce the risk of hyperoxia. Therefore, by initially supplying oxygen only to the first interface region 240a, the oxygenator 200 exposes the blood to a reduced amount of oxygen and thus reduces the risk of over-oxygenating the patient. The oxygenation of the patient can then be adjusted by adjusting the concentration of oxygen in and/or the flow rate of the first oxygenation gas 242a. Then, when it is desired to increase the patient's oxygenation saturation (e.g. during the end of a clinical intervention, such as after surgery to repair a congenital heart defect), the supply of the second oxygenation gas 242b can be initiated to increase the amount of oxygen to which the blood is exposed.

Optionally, the method 500 (e.g. the adjusting 504) may further comprise, responsive to the measured value being below the target value, increasing the amount of oxygen to which the blood is exposed by increasing 508 the concentration of oxygen in and/or the flow rate of the first oxygenation gas 242a.

Optionally, the method 500 may further comprise, after initiating supply of the first oxygenation gas 242a, increasing 510 the concentration of oxygenation in and/or the flow rate of the second oxygenation gas 242b to further increase the amount of oxygen to which the blood is exposed.

FIG. 6 depicts a third example method 600 of controlling blood oxygenation in a patient, as contemplated herein. The controller 150 may be configured to perform any or all of the operations of the method 600. The method 600 may be performed following the method 400 in FIG. 4, or may be performed independently. That is, a controller 150 may be configured to perform the method 400 and/or to perform the method 600.

The method 600 comprises receiving 602 a measured value of a physiological parameter indicative of the level of carbon dioxide in the patient's blood. The measured value may, for example, be measured by the sensor 110 and received by the controller 150 from the sensor 110. The method 600 further comprises calculating 604 a difference between the measured value and the target value of the physiological parameter. The method 600 further comprises supplying 606 the first oxygenation gas 242ato the first interface region 240a of the gas-blood interface 240 and the second oxygenation gas 242b to the second interface region 240b of the gas-blood interface 240. The method 600 further comprises, responsive to the measured value being above the target value, increasing 608 the flow rate of the second oxygenation gas 242b while concurrently reducing the concentration of oxygen in the second oxygenation gas 242b. Following increasing 608 the flow rate of the second oxygenation gas 242b, the method may comprise recalculating the difference between the measured value and the target value, as indicated by the return arrow in FIG. 6. It will be appreciated that various operations within the method 600, and the method 600 as a whole, may be performed repeatedly and/or continuously. For example, the method 600 may comprise receiving updated measured values and repeating the method 600 in response to the updated measured value.

It will be appreciated that the supplying of the first and second oxygenation gases may occur at a different point within the method 600. For example, the first and second oxygenation gases may be supplied before a measured value is received.

Advantageously, the method 600 is particularly beneficial in the treatment of a patient who is experiencing increased levels of carbon dioxide in the blood. For example, during endovascular vein harvesting in coronary artery bypass graft procedures, during resuscitation following a long period of perfusion, or during hypothermic circulatory arrest (in which a patient's metabolic rate is reduced) a patient may experience increased levels of carbon dioxide in the blood. Following these increases of carbon dioxide, the controller 150, being configured to perform method 600, responds by increasing the flow rate of the second oxygenation gas 242b while concurrently reducing the concentration of oxygen in the second oxygenation gas 242b. The increase in flow rate leads to an increased rate of removal of carbon dioxide from the blood, while reducing the concentration of oxygen prevents or reduces the increase of oxygen delivery. Thus, the delivery of oxygen can be maintained while increasing the removal of carbon dioxide from the patient's blood.

Turning to FIG. 7, an example of the operation of the system as described herein will be described. FIG. 7 shows a schematic representation of several ‘states’ that may exist in the operation of the system described herein. In particular, FIG. 7 illustrates a series of states that may occur during treatment of a patient who experiences a low metabolic rate, inadequate carbon dioxide removal, or low oxygen saturation (e.g. cyanosis).

FIG. 7 is divided into a series of four states, (a)-(d), which will be described in sequence below. The transitions between the states are illustrated by arrows 760, 762, 764, and 766. In each state, an oxygenator 700 is depicted. For clarity, the details of oxygenator 700 will not be described and are not labelled in FIG. 7. However, it will be appreciated that oxygenator 700 may share any or all of the features of oxygenator 200, as described above. The only features labelled in FIG. 7 are the first interface region 740a, the second interface region 740b, the first oxygenation gas 742a, and the second oxygenation gas 742b. The following description will begin with state (a), but it will be appreciated that the process is cyclic and so any state may be considered the ‘start’.

In state (a), the first interface region 740a is supplied with the first oxygenation gas 742a. The second interface region 740b is not supplied with oxygenation gas. As depicted in FIG. 7, the first oxygenation gas 742a is supplied with an FiO2 of 100%.

That is, in state (a), the first oxygenation gas 742a consists solely of oxygen. The oxygenator 700 may be operated in this state in order to remove carbon dioxide from the blood of the patient, while also providing oxygen to the blood. In this regard, the first oxygenation gas 742a may be considered a ‘sweep gas’. However, given that the first interface region 740a represents only a proportion (e.g. 40%) of the overall gas-blood interface, the supply of the first oxygenation gas 742a may not be sufficient to reach a target value of a physiological parameter indicative of oxygenation in the patient's blood (e.g. saturation of oxygen). For example, the sensor may measure a measured value of the physiological parameter below the target value. The system may then transition to state (b), as indicated by arrow 760.

In state (b), the first interface region 740a continues to be supplied with the first oxygenation gas 742a, at 100% FiO2. However, the second interface region 740b now begins to be supplied with the second oxygenation gas 742b. That is, the flow rate of the second oxygenation gas 742b may be increased from zero. The flow rate of the second oxygenation gas 742b may continue to be increased until the target value is reached (e.g. until the sensor measures a value equal to the target value). It will be appreciated that supplying the second interface region 740b with the second oxygenation gas 242b increases the amount of oxygen to which the blood is exposed beyond what is possible by only supplying the first interface region 740a. Therefore, a higher saturation of oxygen may be achieved by supplying both interface regions 740a, 740b. The second oxygenation gas 742b may be provided at 100% FiO2 (i.e. pure oxygen). The concentration of oxygen in the second oxygenation gas 742b may be adjusted in addition to or instead of adjusting the flow rate of the second oxygenation gas 742b. The system may operate in this state to increase the saturation of oxygen in the patient's blood to a healthy level (e.g. 98.5%). However, during a surgical procedure, the patient's metabolic rate may drop. This, in turn, means that the blood needs to be exposed to a lower amount of oxygen in the oxygenator to achieve the same saturation of oxygen in the patient's blood. This drop in metabolic rate may be detected in the form of an increase in the saturation of oxygen in the patient's blood caused by the continual supply of the first and second oxygenation gases 742a, 742b at the same oxygen concentration and flow rate in spite of the drop in metabolic rate. This may be detected, for example, by the sensor. In such a case, the system may then transition to state (c), as indicated by arrow 762.

In embodiments in which the system is controlling the level of carbon dioxide in the patient's blood (in addition to, or instead of, the oxygenation of the patient's blood), the concentration of oxygen in the second oxygenation gas 742b may be reduced concurrently with the increase of the flow rate in the second oxygenation gas 742b, while in state (b).

In state (c), the flow rate of the second oxygenation gas 742b is reduced (e.g. to a threshold value, such as zero). This reduces the volume of the second oxygenation gas 742b present in the second interface region 740b at a given time. The amount of oxygen to which the blood is exposed is thus reduced, leading to a decrease in the oxygenation of the patient's blood. The measured value may reduce to the target value at a lower flow rate of the second oxygenation gas 742b. Alternatively, the flow rate of the second oxygenation gas 742b may need to be reduced to zero (such that there is no oxygenation gas in the second interface region 640b) before the target saturation of oxygen can be achieved. In some instances, it may be that this reduction to zero still is not a sufficient reduction to achieve the target saturation of oxygen. Therefore, to continue to reduce the amount of oxygen to which the blood is exposed (e.g. in response to the flow rate of the second oxygenation gas 742b being reduced to zero), the system may then transition to state (d), as indicated by arrow 764.

In state (d), there is no supply of oxygenation gas to the second interface region 740b. To further reduce the amount of oxygen to which the blood is exposed, the concentration of oxygen in and/or the flow rate of the first oxygenation gas 742a may be reduced. This further reduces the amount of oxygen in the oxygenator 700 and thus allows the physiological parameter indicative of oxygenation in the patient's blood to be further reduced until the measured value reaches the target value. Advantageously, this allows the system to effectively manage the level of oxygenation in a patient who is experiencing a low metabolic rate (e.g. a patient in circulatory arrest). As the patient returns to a higher (e.g. normal) metabolic rate, the FiO2 and/or the flow rate of the first oxygenation gas 742a can be increased again to increase the amount of oxygen present in the oxygenator 700. The FiO2 can continue to be increased until it reaches 100%. The system thereby transitions back to state (a), as indicated by arrow 766.

It will be appreciated that FIG. 7 represents a particular example of the operation of the systems described herein in a patient whom initially has a normal metabolic rate and subsequently has a low metabolic rate. In practice, a patient's metabolic rate may increase or decrease during a surgical procedure and thus the system may move freely between all of the states in FIG. 7. That is, the system is not restricted merely to the order described but instead may increase and/or reduce the concentration and/or flow rate of the first and/or second oxygenation gases as needed to achieve the target value of the physiological parameter indicative of the oxygenation of the patient's blood.

As already mentioned, any of the states (a)-(d) in FIG. 7 may be treated as the starting state, depending on the particular clinical scenario.

For example, in the case of method 400, the system may start in state (c), in which both oxygenation gases are being supplied, and the flow rate of the second oxygenation gas 642b is being reduced to a threshold value (e.g. zero). In this case, in state (d), the concentration of oxygen in and/or the flow rate of the first oxygenation gas 742a may be reduced and subsequently increased, as discussed above.

In another example, in the case of method 500, the system may start in state (a), in which only the first oxygenation gas 242a is initially supplied, and the concentration of oxygen in and/or the flow rate of the first oxygenation gas 242a is adjusted.

In another example, in the case of method 600, the system may start in state (b), in which both oxygenation gases are being supplied, and the flow rate of the second oxygenation gas 642b is being increased. In this case, in state (b), the concentration of oxygen in the second oxygenation gas 242b is being concurrently reduced.

It will be understood that any features, functions, characteristics, or advantages described with respect to the above-mentioned examples of a system may be applied to the above-mentioned examples of methods, and vice versa.

In accordance with the present disclosure, the following aspects are also contemplated.

Aspect 1. A method of controlling blood oxygenation in a patient using an oxygenator having a gas-blood interface that is configured to receive blood from the patient and to expose the blood to an amount of oxygen as the blood passes through the oxygenator to produce oxygenated blood, wherein the gas-blood interface comprises a first interface region and a second interface region that are each configured to be independently supplied with a respective oxygenation gas, the method comprising:
receiving a measured value of a physiological parameter indicative of the oxygenation of the patient's blood;
calculating a difference between the measured value and a target value of the physiological parameter;
supplying a first oxygenation gas to the first interface region of the gas-blood interface;
supplying a second oxygenation gas to the second interface region of the gas-blood interface;
responsive to the measured value being above the target value, decreasing the amount of oxygen to which the blood is exposed by reducing the flow rate of the second oxygenation gas to reduce the difference; and
responsive to the flow rate of the second oxygenation gas being reduced to a threshold value, reducing the concentration of oxygen in and/or the flow rate of the first oxygenation gas.

Aspect 2. The method of Aspect 1, further comprising decreasing the amount of oxygen to which the blood is exposed by reducing the concentration of oxygen in the second oxygenation gas.

Aspect 3. The method of Aspect 1 or Aspect 2, further comprising reducing the flow rate of the second oxygenation gas while maintaining a constant concentration of oxygen of oxygen in and/or a constant flow rate of the first oxygenation gas.

Aspect 4. The method of Aspect 3, wherein the constant concentration of oxygen in the first oxygenation gas is 100%.

Aspect 5. The method of any of the preceding Aspects, further comprising maintaining the flow rate of the second oxygenation gas at the threshold value and increasing the amount of oxygen to which the blood is exposed by increasing the concentration of oxygen in and/or the flow rate of the first oxygenation gas.

Aspect 6. The method of any of the preceding Aspects, wherein the threshold value is a flow rate of zero.

Aspect 7. The method of any of the preceding Aspect, further comprising:
initially, solely supplying the first oxygenation gas to the first interface region without supplying the second oxygenation gas to the second interface region;
adjusting the amount of oxygen to which the blood is exposed by adjusting the concentration of oxygen in and/or the flow rate of the first oxygenation gas; and
subsequently, initiating supply of the second oxygenation gas to the second interface region of the gas-blood interface to increase the amount of oxygen to which the blood is exposed.

Aspect 8. The method of Aspect 7, wherein adjusting the amount of oxygen to which the blood is exposed comprises, responsive to the measured value being below the target value, increasing the amount of oxygen to which the blood is exposed by increasing the concentration of oxygen in and/or the flow rate of the first oxygenation gas.

Aspect 9. The method of Aspect 7 or Aspect 8, wherein initiating the supply of the second oxygenation gas is responsive to the concentration of oxygen in the first oxygenation gas having been increased to a threshold concentration.

Aspect 10. The method of any of Aspects 7 to 9, further comprising, after initiating supply of the second oxygenation gas, increasing the concentration of oxygen in and/or the flow rate of the second oxygenation gas to further increase the amount of oxygen to which the blood is exposed.

Aspect 11. The method of any of the preceding Aspects, further comprising:
receiving a second measured value of a second physiological parameter indicative of the level of carbon dioxide in the patient's blood; and
calculating a difference between the second measured value and a second target value of the second physiological parameter.

Aspect 12. the Method of Aspect 11, Further Comprising:
responsive to the second measured value being above the second target value, increasing the flow rate of the second oxygenation gas while concurrently reducing the concentration of oxygen in the second oxygenation gas.

Aspect 13. The method of Aspect 12, wherein increasing the flow rate of the second oxygenation gas while concurrently reducing the concentration of oxygen in the second oxygenation gas maintains a constant volume of oxygen within the second interface region.

Aspect 14. The method of Aspect 12 or Aspect 13, further comprising concurrently maintaining a constant concentration of oxygen in and a constant flow rate of the first oxygenation gas while increasing the flow rate of the second oxygenation gas and concurrently reducing the concentration of oxygen in the second oxygenation gas.

Aspect 15. A method of controlling blood oxygenation in a patient using an oxygenator having a gas-blood interface that is configured to receive blood from the patient and to expose the blood to an amount of oxygen as the blood passes through the oxygenator to produce oxygenated blood, wherein the gas-blood interface comprises a first interface region and a second interface region that are each configured to be independently supplied with a respective oxygenation gas, the method comprising:
receiving a measured value of a physiological parameter indicative of the level of carbon dioxide in the patient's blood;
calculating a difference between the measured value and a target value of the physiological parameter;
supplying a first oxygenation gas to the first interface region of the gas-blood interface;
supplying a second oxygenation gas to the second interface region of the gas-blood interface;
responsive to the measured value being above the target value, increasing the flow rate of the second oxygenation gas while concurrently reducing the concentration of oxygen in the second oxygenation gas.

Aspect 16. The method of Aspect 15, comprising increasing the flow rate of the second oxygenation gas while concurrently reducing the concentration of oxygen in the second oxygenation gas so as to maintain a constant volume of oxygen within the second interface region.

Aspect 17. The method of Aspect 15 or Aspect 16, further comprising maintaining the flow rate and/or the concentration of the first oxygenation gas while increasing the flow rate of the second oxygenation gas and concurrently reducing the concentration of oxygen in the second oxygenation gas.

1. A system for controlling blood oxygenation in a patient using an oxygenator having a gas-blood interface that is configured to receive blood from the patient and to expose the blood to an amount of oxygen as the blood passes through the oxygenator to produce oxygenated blood, wherein the gas-blood interface comprises a first interface region and a second interface region that are each configured to be independently supplied with a respective oxygenation gas, the system comprising:
a controller configured to:
receive a measured value of a physiological parameter indicative of the oxygenation of the patient's blood and calculate a difference between the measured value and a target value of the physiological parameter; and
control a gas supply apparatus to
supply the first oxygenation gas to the first interface region and supply the second oxygenation gas to the second interface region,
responsive to the measured value being above the target value, decrease the amount of oxygen to which the blood is exposed by reducing the flow rate of the second oxygenation gas to reduce the difference, and
responsive to the flow rate of the second oxygenation gas being reduced to a threshold value, reduce the concentration of oxygen in and/or the flow rate of the first oxygenation gas.

2. The system of claim 1, wherein the controller is further configured to control the gas supply apparatus to decrease the amount of oxygen to which the blood is exposed by reducing the concentration of oxygen in the second oxygenation gas.

3. The system of claim 1, wherein the controller is configured to control the gas supply apparatus to reduce the flow rate of the second oxygenation gas while maintaining a constant concentration of oxygen in and/or a constant flow rate of the first oxygenation gas.

4. The system of claim 3, wherein the constant concentration of oxygen in the first oxygenation gas is 100%.

5. The system of claim 1, wherein the controller is further configured to control the gas supply apparatus to:
maintain the flow rate of the second oxygenation gas at the threshold value and increase the amount of oxygen to which the blood is exposed by increasing the concentration of oxygen in and/or the flow rate of the first oxygenation gas.

6. The system claim 1, wherein the threshold value is a flow rate of zero.

7. The system of claim 1, wherein the controller is further configured to control the gas supply apparatus to:
initially, solely supply the first oxygenation gas to the first interface region without supplying the second oxygenation gas to the second interface region;
adjust the amount of oxygen to which the blood is exposed by adjusting the concentration of oxygen in and/or the flow rate of the first oxygenation gas; and
subsequently, initiate supply of the second oxygenation gas to the second interface region of the gas-blood interface to increase the amount of oxygen to which the blood is exposed.

8. The system of claim 7, wherein the controller is configured to control the gas supply apparatus to adjust the amount of oxygen to which the blood is exposed by, responsive to the measured value being below the target value, increasing the amount of oxygen to which the blood is exposed by increasing the concentration of oxygen in and/or the flow rate of the first oxygenation gas.

9. The system of claim 7, wherein the controller is configured to control the gas supply apparatus to initiate the supply of the second oxygenation gas responsive to the concentration of oxygen in the first oxygenation gas having been increased to a threshold concentration.

10. The system of claim 7, wherein the controller is further configured to, after initiating supply of the second oxygenation gas, increase the concentration of oxygen in and/or the flow rate of the second oxygenation gas to further increase the amount of oxygen to which the blood is exposed.

11. The system of claim 1, wherein the controller is further configured to:
receive a second measured value of a second physiological parameter indicative of the level of carbon dioxide in the patient's blood; and
calculate a difference between the second measured value and a second target value of the second physiological parameter.

12. The system of claim 11, wherein the controller is further configured to control the gas supply apparatus to:
responsive to the second measured value being above the second target value, increase the flow rate of the second oxygenation gas while concurrently reducing the concentration of oxygen in the second oxygenation gas.

13. The system of claim 12, wherein the controller is configured to control the gas supply apparatus to increase the flow rate of the second oxygenation gas while concurrently reducing the concentration of oxygen in the second oxygenation gas so as to maintain a constant volume of oxygen within the second interface region.

14. The system of claim 12, wherein the controller is configured to control the gas supply apparatus to concurrently maintain a constant concentration of oxygen in and a constant flow rate of the first oxygenation gas while increasing the flow rate of the second oxygenation gas and concurrently reducing the concentration of oxygen in the second oxygenation gas.

15. A system for controlling blood oxygenation in a patient using an oxygenator having a gas-blood interface that is configured to receive blood from the patient and to expose the blood to an amount of oxygen as the blood passes through the oxygenator to produce oxygenated blood, wherein the gas-blood interface comprises a first interface region and a second interface region that are each configured to be independently supplied with a respective oxygenation gas, the system comprising:
a controller configured to:
receive a measured value of a physiological parameter indicative of the level of carbon dioxide in the patient's blood and calculate a difference between the measured value and a target value of the physiological parameter; and
control a gas supply apparatus to
supply the first oxygenation gas to the first interface region and supply the second oxygenation gas to the second interface region,
responsive to the measured value being above the target value, increase the flow rate of the second oxygenation gas while concurrently reducing the concentration of oxygen in the second oxygenation gas.

16. The system of claim 15, wherein the controller is configured to control the gas supply apparatus to increase the flow rate of the second oxygenation gas while concurrently reducing the concentration of oxygen in the second oxygenation gas so as to maintain a constant volume of oxygen within the second interface region.

17. The system of claim 15, wherein the controller is configured to control the gas supply apparatus to concurrently maintain a constant flow rate and/or a constant concentration of the first oxygenation gas while increasing the flow rate of the second oxygenation gas and concurrently reducing the concentration of oxygen in the second oxygenation gas.

18. The system of claim 1, wherein the system further comprises the gas supply apparatus, the gas supply apparatus being configured to control the flow rate of and/or the concentration of oxygen in the first oxygenation gas and the second oxygenation gas.

19. The system of claim 1, wherein the system further comprises the oxygenator,
wherein the oxygenator comprises a gas inlet zone for receiving oxygenation gas into the gas-blood interface, wherein the gas inlet zone comprises a partition dividing the gas inlet zone into:
a first gas inlet region configured to receive the first oxygenation gas and to provide the first oxygenation gas to the first interface region; and
a second gas inlet region configured to receive the second oxygenation gas and to provide the second oxygenation gas to the second interface region.

20. (canceled)

21. The system of claim 1, wherein the system further comprises a sensor configured to measure the physiological parameter, and wherein the controller is configured to receive the measured value from the sensor.

22.-23. (canceled)
